(19) United States
(12) Plant Patent Application Publication
(10) Pub. No.: US 2026/0090488 P1
(43) Pub. Date: Mar. 26, 2026

(54) Boxwood Plant Named 'EADS1'

(50) ;

(71) Applicant: Saunders Genetics, LLC, Piney River, VA (US)

(72) Inventor: Dale EADS, Loveland, OH (US)

(73) Assignee: Saunders Genetics, LLC, Piney River, VA (US)

(21) Appl. No.: 19/332,777

(22) Filed: Sep. 18, 2025

Related U.S. Application Data
(60) Provisional application No. 63/696,933, filed on Sep. 20, 2024.

(51) Int. Cl. A01H 6/00; A01H 5/12

(52) U.S. Cl. CPC A01H 6/00; A01H 5/12
USPC PLT/226

(57) ABSTRACT

A new and distinct variety of boxwood plant, referred to by its cultivar name, ‘EADS1’, is disclosed. The new variety provides olive green leaf color. Smaller leaves are displayed. Very good resistance to box tree moth is exhibited. The growth habit shape is typical of boxwood varieties. The new variety is well suited for providing ornamentation in the landscape.

Latin name of genus and species of plant claimed: Buxus sinica variety insularis.

Variety denomination: ‘EADS1’.

### BACKGROUND OF THE INVENTION

The new variety of boxwood plant was discovered growing in a cultivated area in Loveland, Ohio.

The new variety has been found to undergo asexual propagation. Asexual propagation has shown that the characteristics of the new variety are stable and are strictly transmissible by such asexual propagation from one generation to another. Accordingly, the new variety undergoes asexual propagation in a true-to-type manner.

### SUMMARY OF THE INVENTION

It was found that the new variety of boxwood plant of the present invention:
(a) displays long internodes,
(b) provides smaller leaves, which are olive green in color, and
(c) exhibits very good resistance to box tree moth.

The new variety well meets the needs of the horticultural industry. It can be grown to advantage as ornamentation in parks, gardens, public areas, and in residential settings. Accordingly, the plant is particularly well suited for growing in the landscape.

The new variety of the present invention can readily be distinguished from similar varieties that are commercially available.

The new variety has been named ‘EADS1’.

### BRIEF DESCRIPTION OF THE PHOTOGRAPH

The accompanying photographs show as nearly true as it is reasonably possible to make the same, in a color illustration of this character, typical specimens of the plant and plant parts of the new variety. Colors in the photographs may differ slightly from the color values cited in the detailed description, which accurately describes the colors of the ‘EADS1’ variety.

FIG. 1—displays the new variety in a hedge.

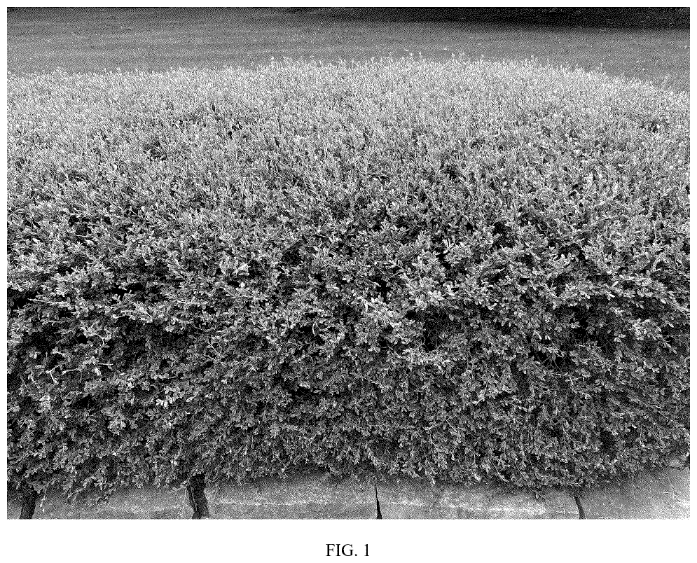

FIG. 2—displays a seedling of the new variety.

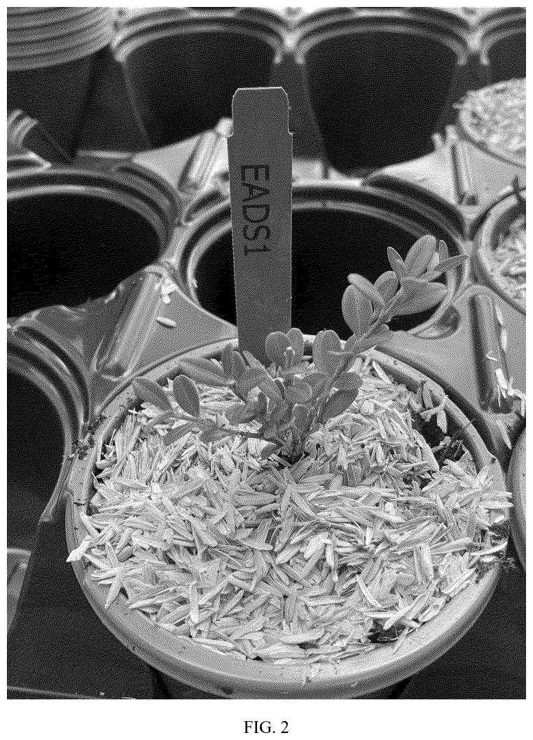

### DETAILED BOTANICAL DESCRIPTION

Botanical classification: Buxus microphylla cultivar ‘EADS1’:
Plant:
   habit.—typical boxwood shape.
Leaves:
   size.—small.
   internode length.—long.
   color.—olive green.
Development:
   tolerance to.—
   disease and pest.—very good resistance to box tree moth.

The new boxwood plant variety ‘EADS1’ has not been observed under all possible environmental conditions to date. Accordingly, it is possible that the phenotypic expression may vary somewhat with changes in light intensity and duration, cultural practices, and other environmental conditions.

I claim:

1. A new and distinct variety of boxwood plant named ‘EADS1’ substantially as herein shown and described.
